(19) United States
(12) Patent Application Publication
(10) Pub. No.: US 2025/0081972 A1
(43) Pub. Date: Mar. 13, 2025

(54) Method Of Using/Applying A Keratin Hydrolysis Peptide Solution Upon Grape Plants To Improve The Color, Flavor, And Sweetness

(71) Applicant: CH Biotech R&D Co., Ltd., Nantou City (TW)

(72) Inventors: Kai XIA, Nantou (TW); Ching-Chi TSAI, Taichung (TW); Meng-Ying LI, Nantou (TW); Yu-Lun LIU, Taichung (TW); Nai-Hua YE, Changhua (TW); Jie-Chao YOU, New Taipei City (TW)

(73) Assignee: CH Biotech R&D Co., Ltd., Nantou City (TW)

(21) Appl. No.: 18/389,780

(22) Filed: Dec. 20, 2023

(30) Foreign Application Priority Data
Sep. 8, 2023 (TW) 112134394
Sep. 11, 2023 (TW) 112134549
Sep. 11, 2023 (TW) 112134551

(51) Int. Cl. A01N 63/50; G01N 33/68

(52) U.S. Cl. CPC A01N 63/50; G01N 33/6848

(57) ABSTRACT

Present invention teaches the method of using a keratin hydrolysis peptide (“KHP”) solution to increase the grape plant's color changing (ripening) rate, flavor and sweetness. By selectively choosing specific weights of feathers and water, and treating the mixture, though one embodiment does not have water mixed in, to a high-temperature high-pressure hydrolysis process, the resulting solution is confirmed to contain at least 253 peptides and then applied to the leaf surface of grape plants at different growth stages. Optionally, the KHP solution can be diluted by water, as taught in the specification, before applying to the leaf surface of the grape plants.

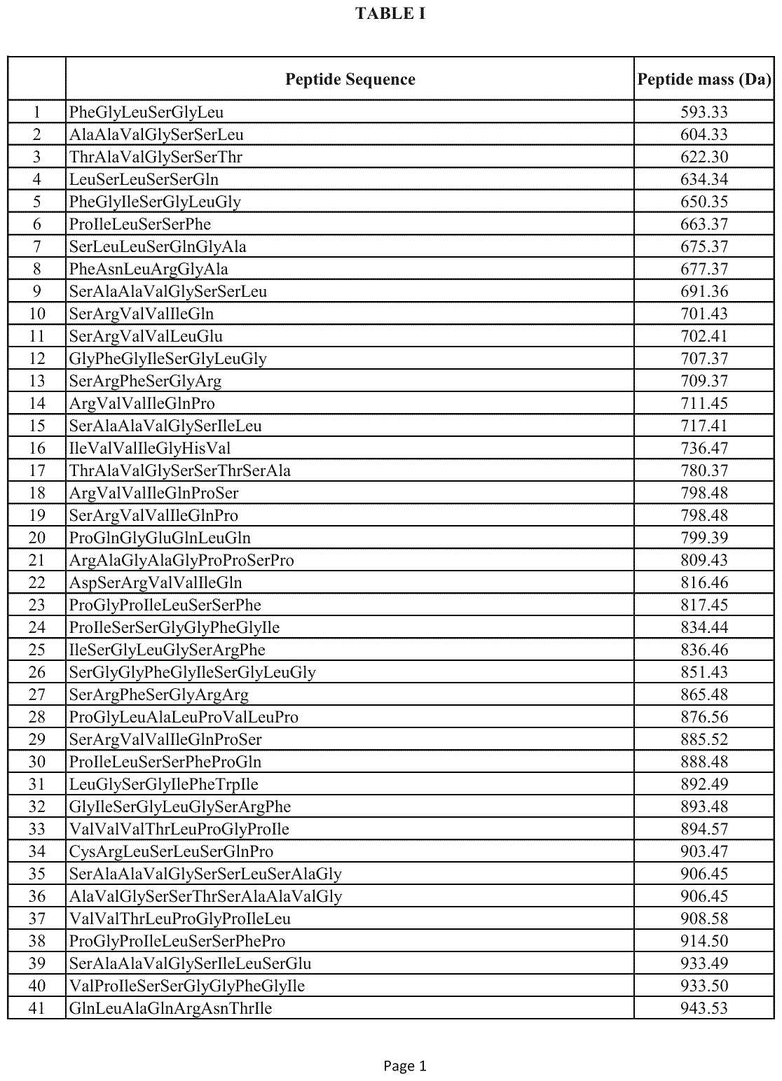

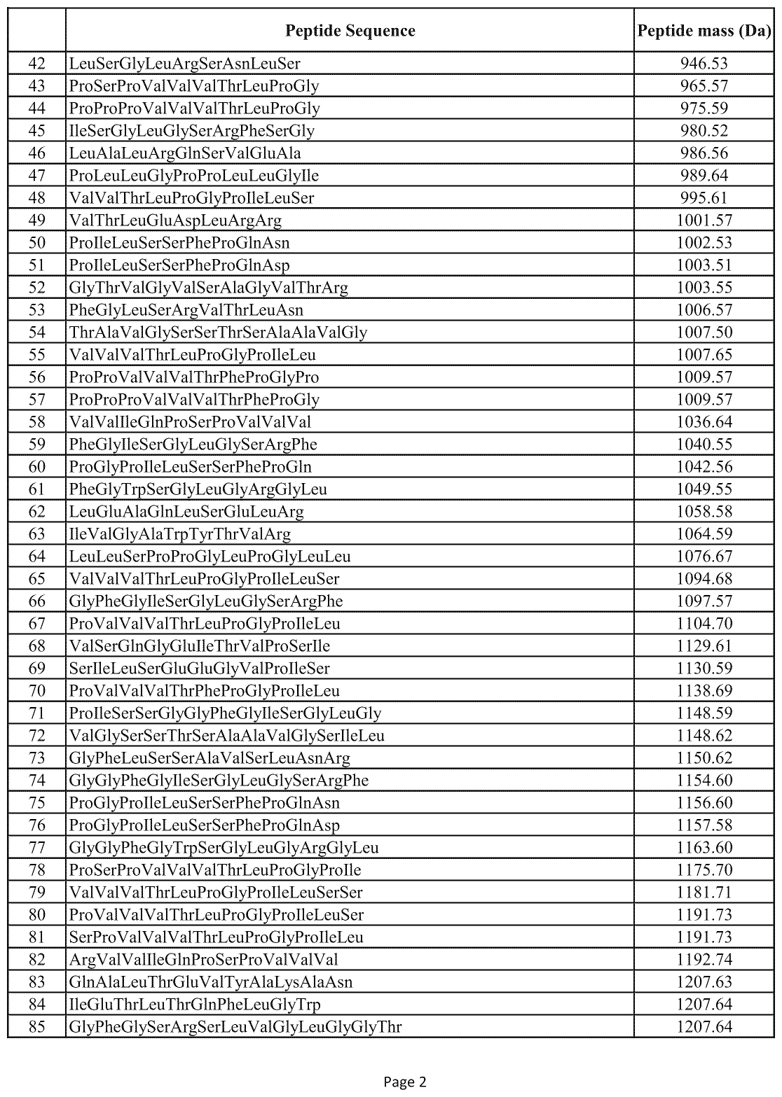

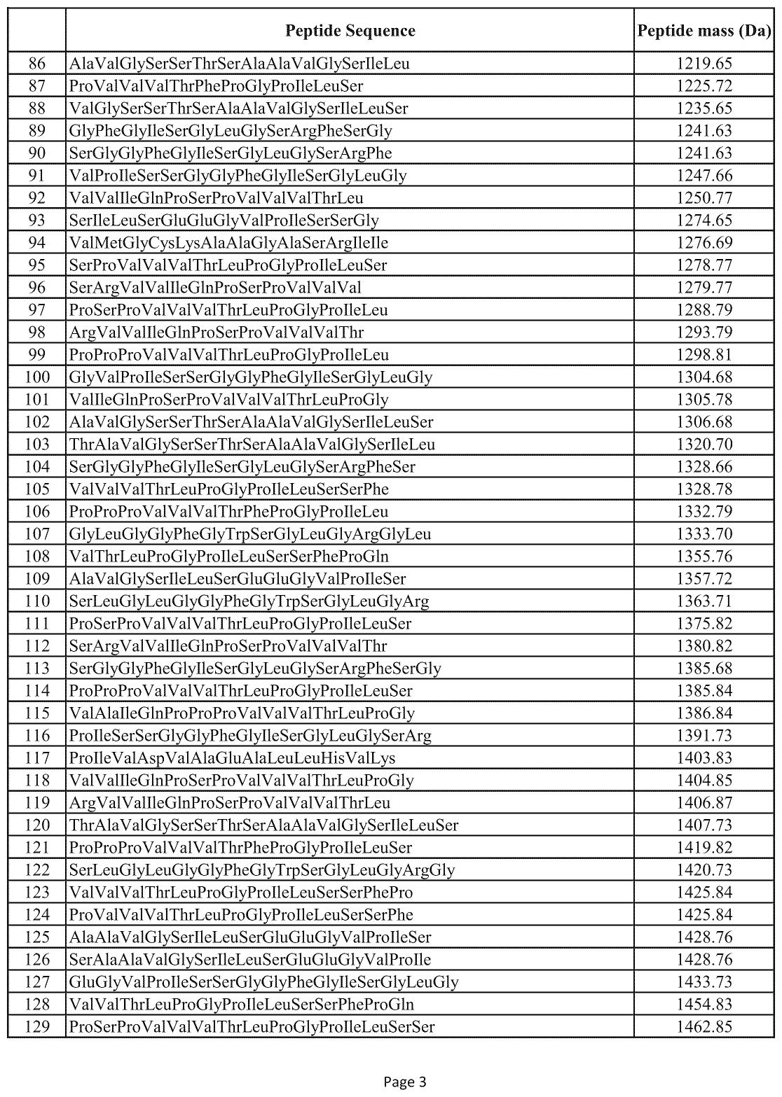

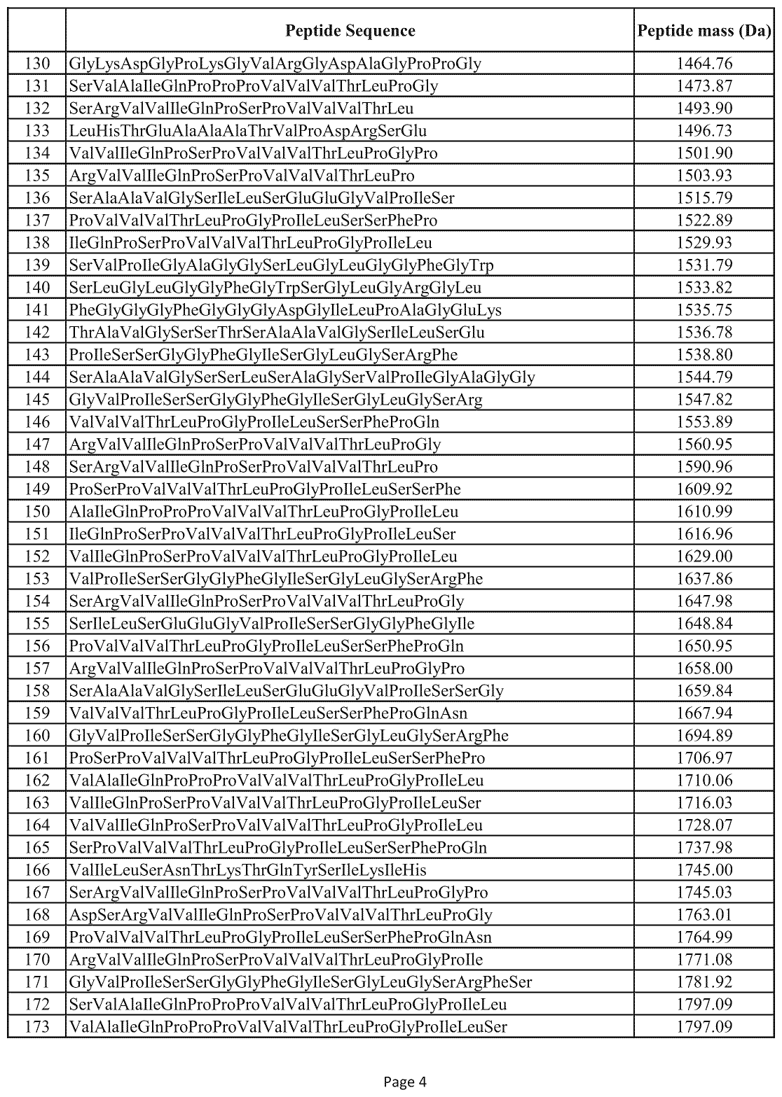

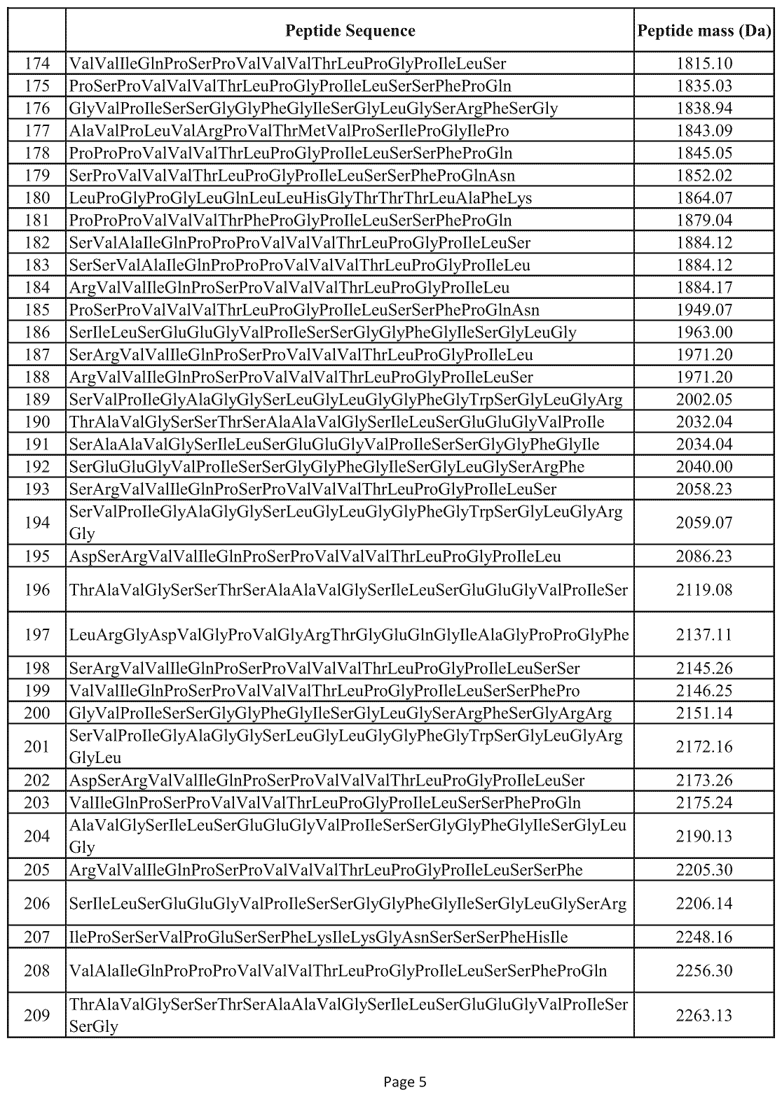

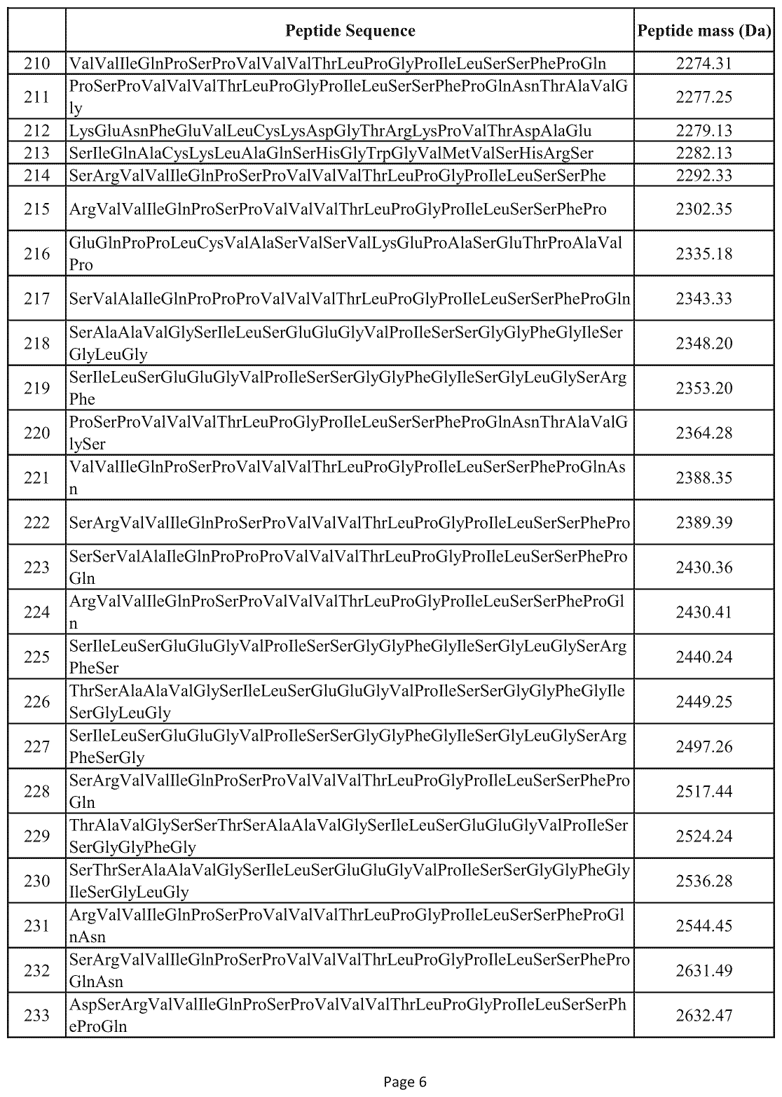

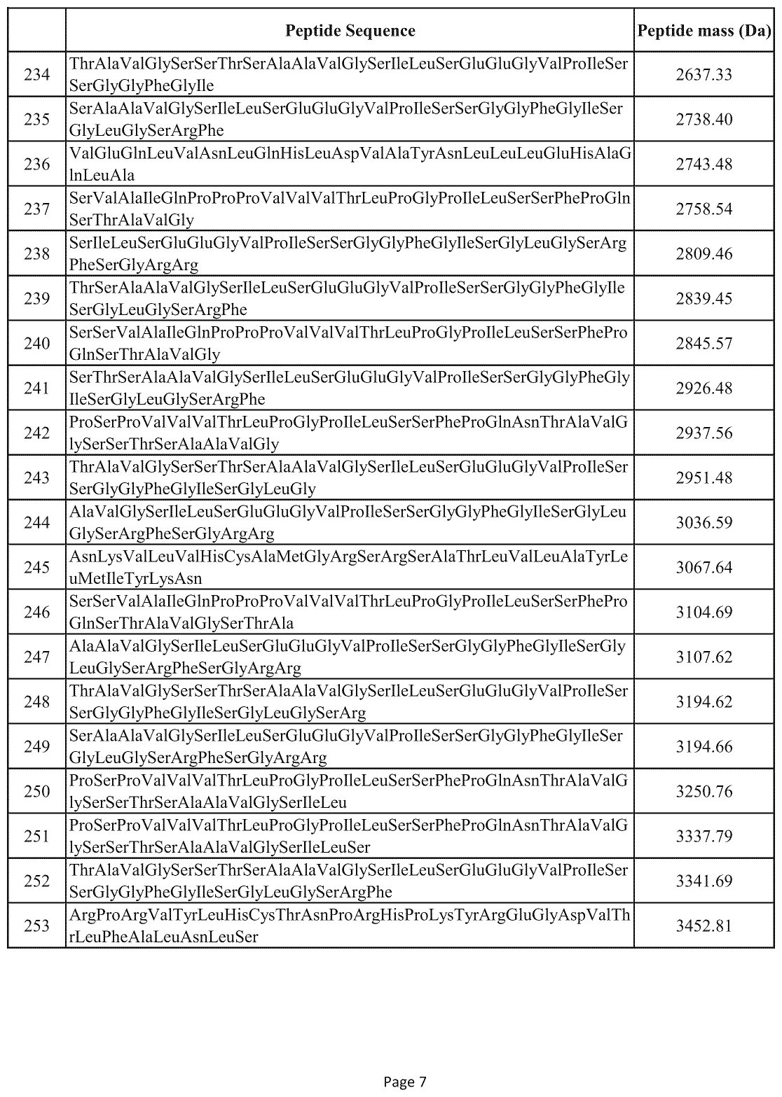

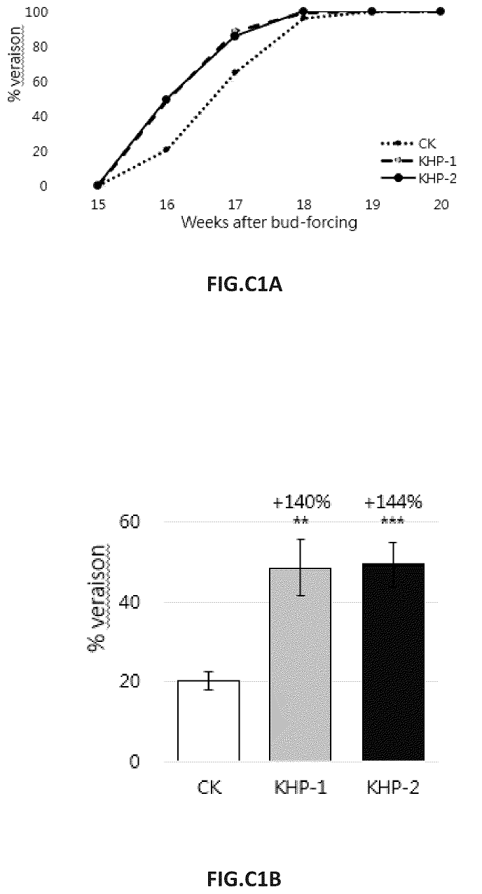

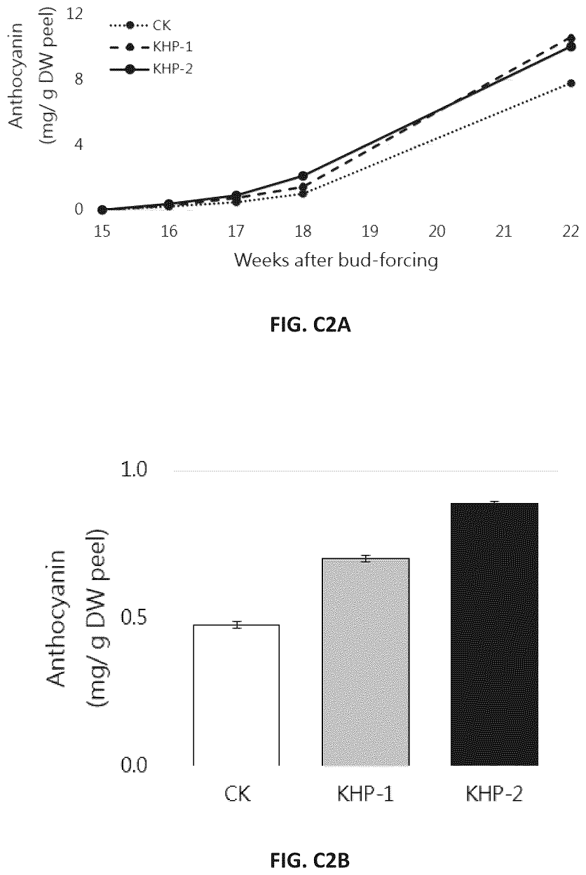

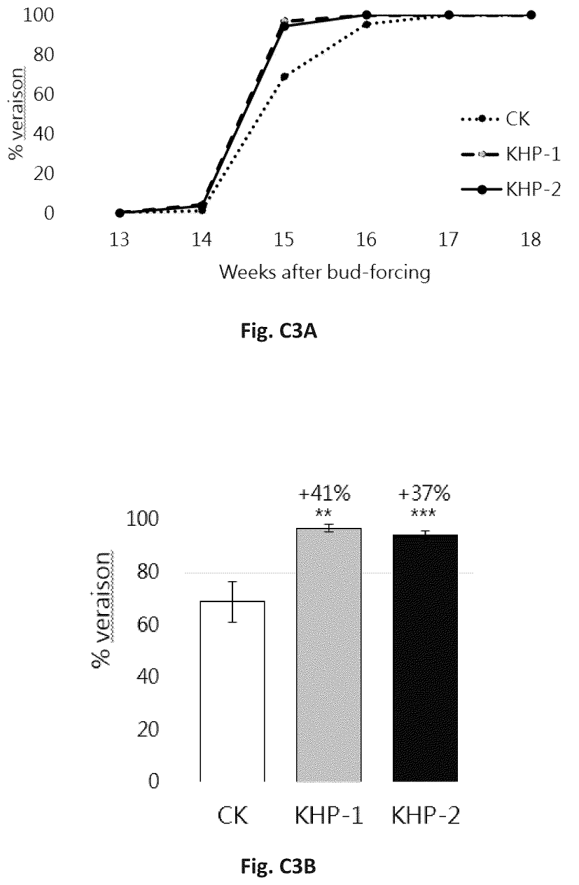

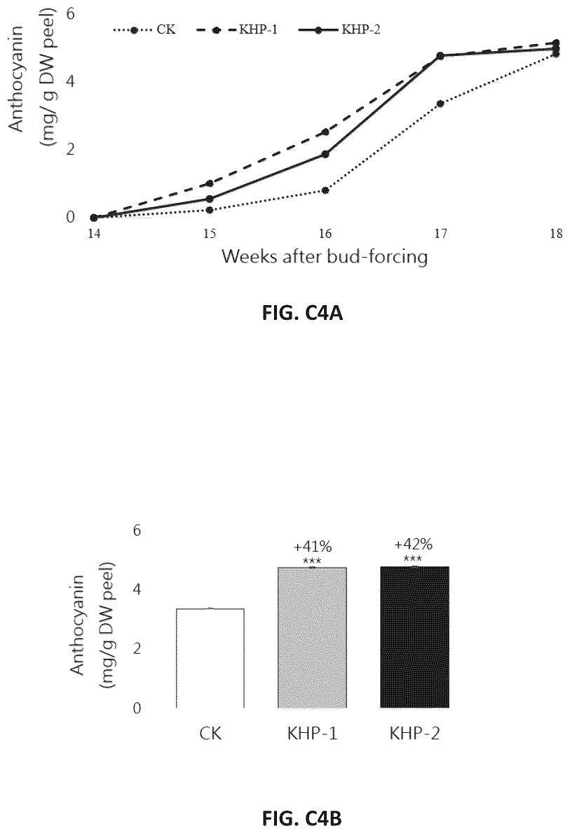

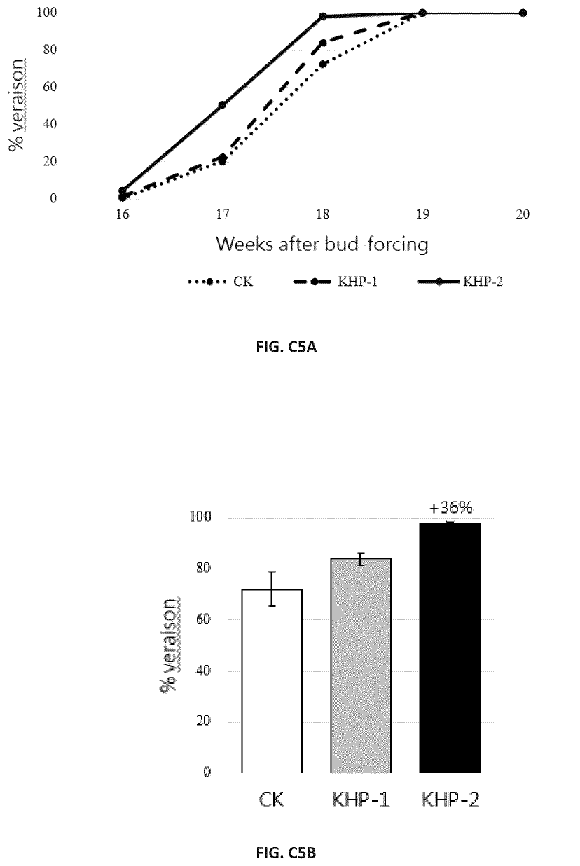

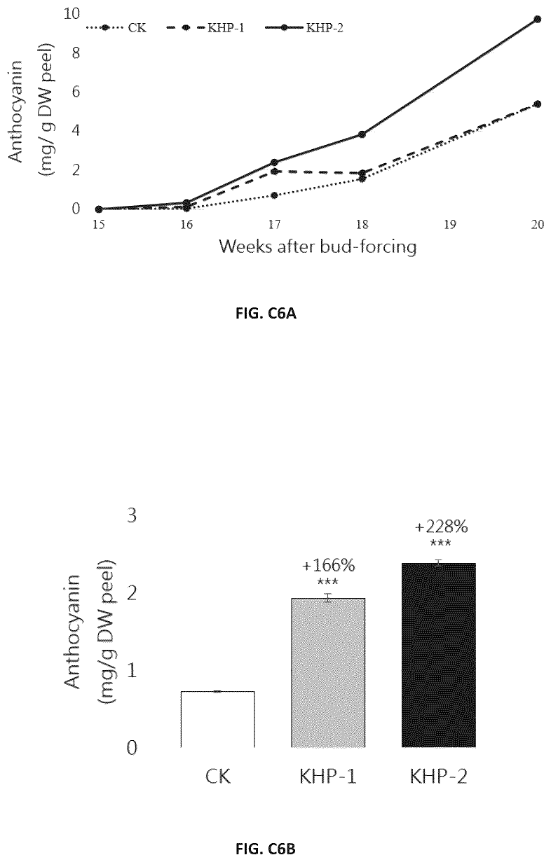

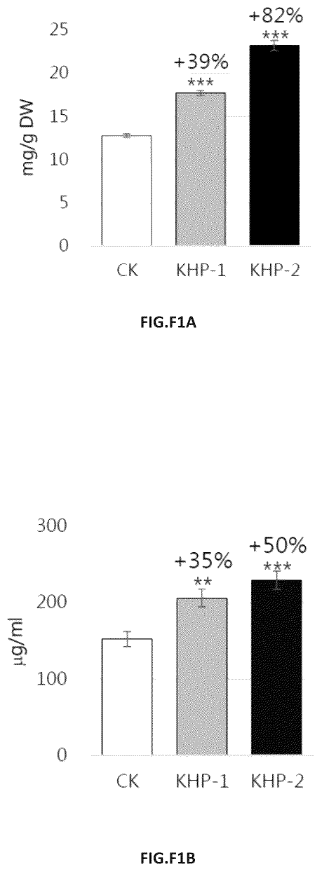

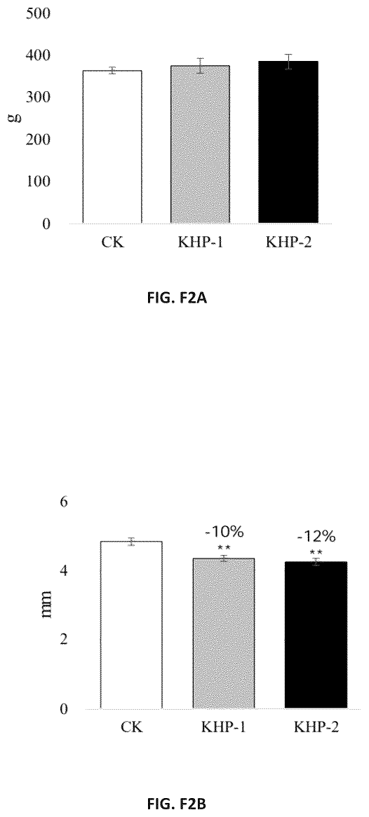

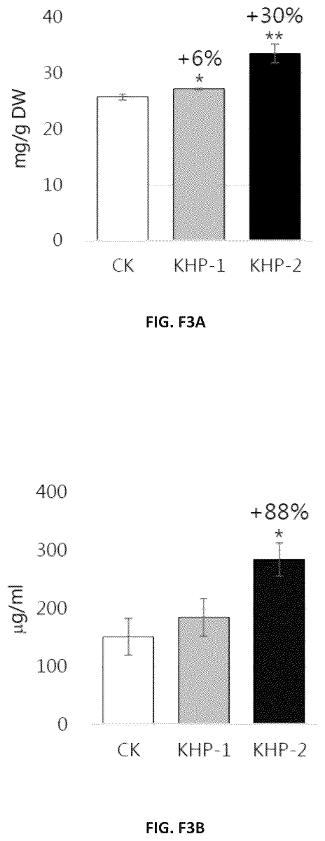

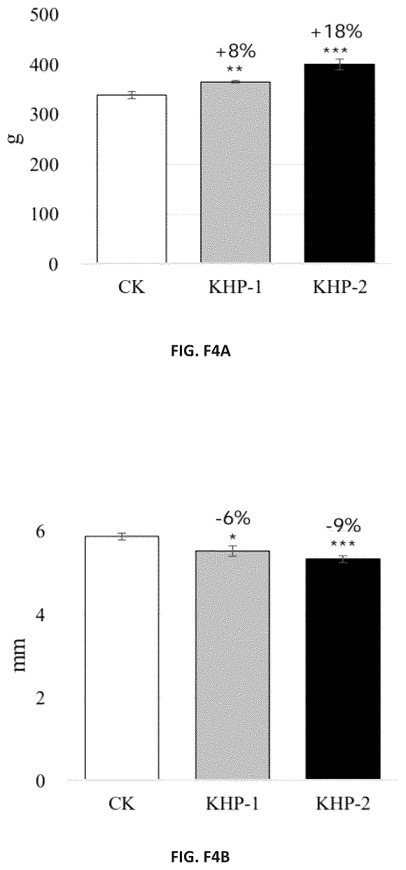

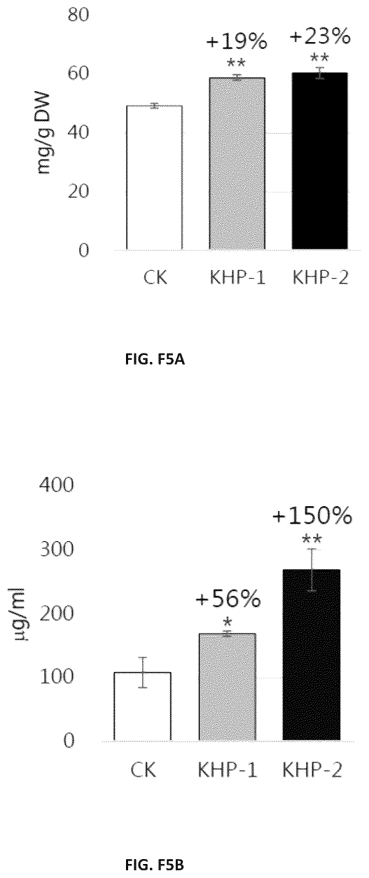

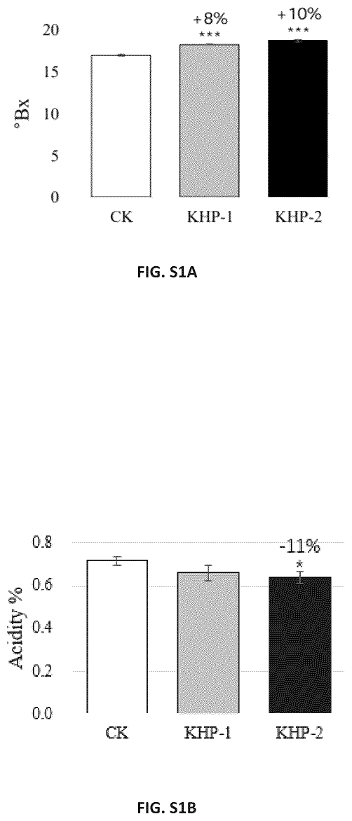

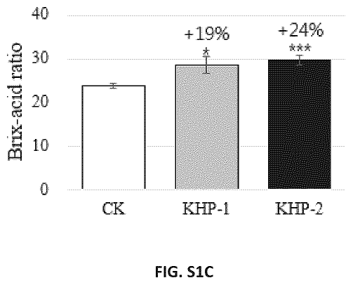

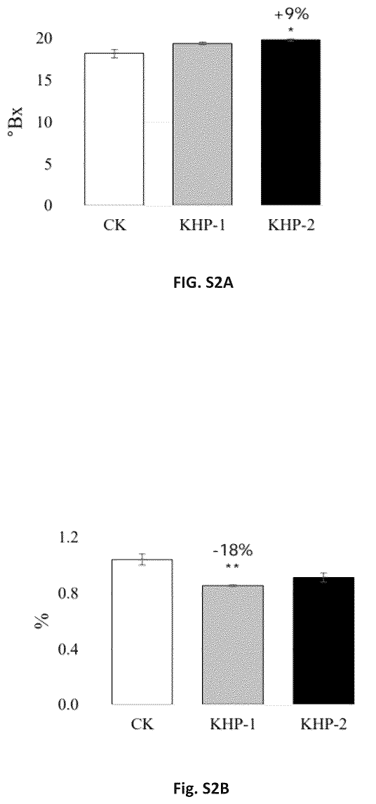

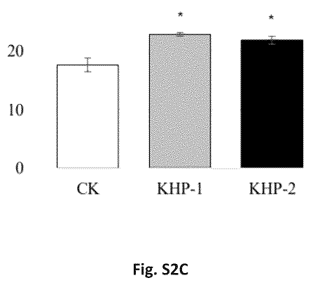

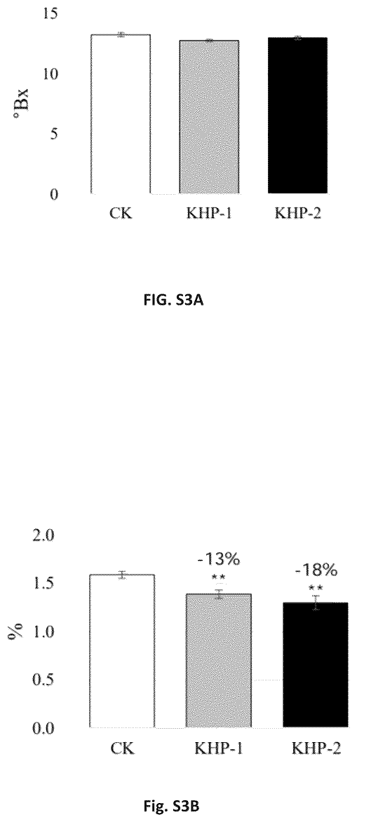

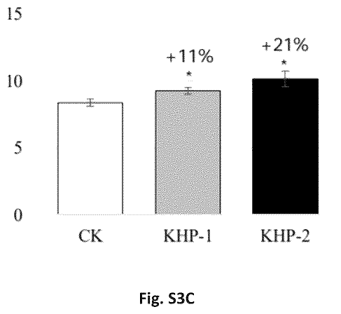

### PRIORITY CLAIM TO FOREIGN APPLICATION

Applicant hereby makes priority claim to Taiwan applications, number 112134394, having the Taiwan filing date of Sep. 8, 2023; number 112134551, having the Taiwan filing date of Sep. 11, 2023, and 112134549, having the Taiwan filing date of Sep. 11, 2023.

### BACKGROUND OF THE INVENTION

Present invention disclosed and claimed the method and application of a keratin hydrolysis peptide (“KHP”) solution to grape plants, improving the grapes' color changing (ripening), flavor and sweetness.

The KHP solution is made by a hydrolysis process using feathers and water, with one version of the solution using feathers only, via a high-temperature and high-pressure process, resulting in a solution that is believed to have potential beneficial uses.

Hydrolyzed keratin has long been used to strengthen hairs, reduce breakage, and minimize damages. Some form of chemical process is used to break down the large protein molecule of the hydrolyzed keratin so that it can penetrate the hair cuticles and do its magic. Such hydrolyzed keratin solution is also known to have some beneficial uses for agricultural applications. However, there are different specific processes of making the keratin solution, based upon different components, and different application steps, leading to different results.

A consensus is being formed that, instead of relying on chemical fertilizers to improve the desired attributes of grapes, such as sweetness and flavors, using natural materials such as feathers, is recognized as a better alternative than the potential environment problems posed by chemical fertilizers.

Grape is one of the largest fruit crops in the world. According to statistics, the global harvesting area reached 6,729,198 metric acers, with total production weight reaching 25 million metric tons.

Grape plants are generally divided into two categories: for direct consumption and for making wines. The grapes grown for direct consumption have been known to be more susceptible to weather changes, over exposure to sun (or under exposure), and, in recent years, the over application of fertilizers that, despite promoting the growth of leaves and foliage in general, lead to the reduction of actual yield.

The Taiwan grape plants' growth and production sometimes has an addition problem: incessant rains the deprived the plants of sufficient sun light for photosynthesis, which further lead to health issues and diseases inflicted upon grape plants.

For harvested grapes, better “looks” always commend a higher price, especially among the species with red or black skin colors. The “looks” include the color change showing ripening status, as well as the crispness of the harvest grapes. As is already known, the grape fruit's skin color is primarily decided by the existence of anthocyanin. The growth conditions, temperature, light, water, nutrients in soil, etc., all affect the production of high-quality grape fruits and the amount of anthocyanin at the right time.

It is also known that the phenolic content directly affects the flavor of grape fruits. In addition, the Total Soluble Solid (TSS) and acidity also affect the taste and flavor of the harvested grape fruits.

Moreover, it is also known that people prefer grapes with high Brix number, which is a percentage of sugar by mass, usually measured in grams, and reflects, indirectly, the health status of a fruit. Fruits tested to have higher Brix number tend to have better overall sweetness and command better sales prices.

For the direct consumption type grapes, the preferred range of Brix number is between 18 and 28, with 0.4-0.6% acidity. For wine-making grapes, the Brix number range is also preferred to be not far off from this range.

Given all the desired attributes, the inventors of present application engaged in the suitable hydrolysis process, using selected components of feathers and water, and conducted field tests to confirm the effectiveness of the KHP solution applications disclosed and claimed herein.

It is well known that feathers of domestic birds/fowls contain 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by mixing feathers and water (generally at a rate of 1:3) for treatment of a high-temperature and high-pressure (setting of 160° C., 6.1 kg/cm², or setting of 180° C. and 9.2 kg/cm² for a duration of 30 minutes) that obtained a solution. The keratin solution is then mixed with other fertilizers containing nitrogen, potassium and other minerals before applying to plants.

The keratin hydrolysis solution is shown to boost the growth of Pogostemon cablin and Vigna radiata, as reported by Nurdiawati et al., 2019.

Under the aegis of CH Biotech, inventors of present application experimented and worked on the method of employing a high-temperature and high-pressure hydrolysis process, materially different from Nurdiawati, to break down the keratin in feathers, and to form a hydrolyzed peptide (“KHP”) solution that can be applied to grape plants, suitable for the purposes as intended herein, increasing the more cost-effective grape farming and harvesting.

### SUMMARY OF THE INVENTION

The invention has two embodiments to create the KHP solution, where one version contains the water:feather weight ratio of 4:5, before mixing and placed into a sealed contained to go through the hydrolysis process. The second version does not add water, but simply uses feathers.

The first embodiment of the KHP solution is made by the steps of:
a. Preparing the KHP solution by mixing 50 kg of feathers whose content is 50% water and 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to leaf surface of the grape plant at the fruit expansion stage.

The confirmation of some of the bioactive 253 peptides is further done by referencing the BIOPEP-UWM database.

The method of using a keratin hydrolysis peptide (KHP) solution stated above where the solution is applied at the growth stage of fruit thinning.

The method of using a KHP solution is further diluted with water by volume at the ratio of 1:250-1,000 or more specifically at 1:300-500, and is then sprayed to the grape plant leaves' surface at the growth stages as taught herein.

A second embodiment of the method of using a keratin hydrolysis peptide (KHP) solution comprising the steps of:
a. Preparing the KHP solution by preparing and putting 70 kg of feathers whose content is 46% water in a sealed container;
b. hydrolyzing the mixture in the container with a temperature and pressure setting of 180° C. and 13 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to the leaf surface of the grape plant at the early budding and/or fruit expansion stage.

The confirmation of some of the bioactive 253 peptides is further done by referencing the BIOPEP-UWM database.

The method of using a keratin hydrolysis peptide (KHP) solution stated above is to apply to the leaf surface of the grape plant at the blossoming and/or fruit thinning stage.

Similar to the first embodiment, the method of using a KHP solution can be further diluted with water by volume at the ratio of 1:250-1,000 or more specifically at the ratio of 1:300-500, at the stages as taught herein.

To prove the efficacy of the method of increasing grape production yield, the inventors conducted experiments in the field and presented the results herein.

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

Table I shows the at least 253 peptides and its annotated sequences for the solution generated in accordance with the disclosure of this application.

FIG. C1A shows the color changing (ripening) status of the 3 groups from week 15 to week 20 after Kyoho grapes were applied with the solutions.

FIG. C1B shows the color changing (ripening) status of the 3 groups at the 16^{th} week.

FIG. C2A shows the color presentation, the anthocyanin content, from the skins of the grape fruits from week 15 to week 22.

FIG. C2B shows the anthocyanin content of the 3 groups at the 17^{th} week.

FIG. C3A shows the color changing status of the 3 groups from week 13 to week 18 after Chunfeng grapes were applied with the solutions.

FIG. C3B shows the color changing status of the 3 groups of Chunfeng grapes at 15^{th} week.

FIG. C4A shows the color representation, the anthocyanin content, from the skins of the Chunfeng grape in the 3 groups from week 14 to week 18.

FIG. C4B shows the anthocyanin content of the Chenfeng grape at week 17.

FIG. C5A shows the color changing status of the 3 groups from the skins of the Black Queen grape from week 16 to week 20.

FIG. C5B shows the color changing status of the 3 groups at week 18.

FIG. C6A shows the anthocyanin content of the Black Queen grape (measured from the skins taken) from week 15 to week 20.

FIG. C6B shows the anthocyanin content of the Black Queen grape at week 17.

FIG. F1A shows the phenolic content taken from Kyoho grapes' skin.

FIG. F1B shows the phenolic content taken from Kyoho grapes juice.

FIG. F2A shows the crispness reading measured from Kyoho grape's skin.

FIG. F2B shows the elasticity reading measured from Kyoho grape's skin.

FIG. F3A shows the phenolic content taken from Chunfeng grape's skin.

FIG. F3B shows the phenolic content taken from Chunfeng grape's juice.

FIG. F4A shows the crispness reading measure from Chunfeng grape's skin.

FIG. F4B shows the elasticity reading measured from Chunfeng grape's skin.

FIG. F5A shows the phenolic content taken from Black Queen grape's skin.

FIG. F5B shows the phenolic content taken from Black Queen grape's juice.

FIGS. S1[A/B/C] show the Brix number, the acidity number and the Brix/Acidity ratio of Kyoho grapes around harvest time.

FIGS. S2[A/B/c] show the Brix number, the acidity number and the Brix/Acidity ratio of Chunfeng grapes around harvest time.

FIGS. S3[A/B/C) show the Brix number, the acidity number and the Brix/Acidity ratio of Black Queen grapes around harvest time.

### DETAILED DESCRIPTION OF THE INVENTION

The method of using a keratin hydrolysis peptide (KHP) solution to improve grape plant's production yield, comprising the steps of:
a. mixing 40 kg of water and 50 kg of feathers together, wherein the feathers are of 50% water content;
b. stirring the feathers and water mixture in a sealed container;
c. hydrolyzing the mixture in the container with a temperature and pressure setting of 185° C. and 12 kg/cm² for a duration of 80 minutes;
d. using a mass spectrometer to confirm the combination of peptides in the solution to contain at least 253 peptides as listed in the specification whereby their molecular masses are between 500 and 4,000 Dalton, and consisting of between 5 and 30 amino acids.
e. the solution is further filtered and concentrated to the range of 300,000-450,000 ppm concentration.
f. Applying water to dilute the solution to grape leaves' surface during a grape plant's early budding and/or fruit expansion stage.

The confirmation of some of the bioactive 253 peptides is further done by referencing the BIOPEP-UWM database.

A second embodiment of the KHP solution is done by using feathers only, without water, by the steps of:
a. putting 70 kg of feathers in a sealed container, wherein the feathers are of 46% water content;
b. hydrolyzing the feathers in the container with a temperature and pressure setting of 180° C. and 13 kg/cm² for a duration of 40 minutes;
c. using a mass spectrometer to confirm the combination of peptides in the solution to contain at least 253 peptides as listed in the specification whereby their molecular masses are between 500 and 4,000 Dalton, and consisting of between 5 and 30 amino acids.
d. the solution is further filtered and concentrated to the range of 300,000-450,000 ppm concentration.
e. Applying water to dilute the solution to grape leaves' surface during a grape plant's expansion stage

The confirmation of some of the bioactive 253 peptides is further done by referencing the BIOPEP-UWM database.

The method of using a keratin hydrolysis peptide (KHP) solution above where the solution may be applied to the leaves' surface at fruit thinning stage.

The method of using a KHP solution above where the solution can be diluted with water by volume at the ratio of 1:250-1,000 and sprayed to the leaves' surface at the growth stages fruit expansion and/or fruit thinning.

The method of using a keratin hydrolysis peptide (KHP) solution above where the solution can be more specifically diluted with water by volume at the ratio of 1:300-500 and sprayed to the leaves' surface at the growth stages of fruit expansion and/or fruit thinning.

To prove the efficacy of the method of increasing grape production yield, the inventors conducted experiments in the field and presented the results herein.

Three groups of grape plants were tested, noted as CK group, KHP-1 group and KHP-2 group. The CK group is the check group where no KHP solution is applied. The other two groups are applied with the first and second embodiment KHP solutions, diluted at 1:500 ratio. The tests went on for about six (6) months. first embodiment solution is noted as KHP-1 and the second embodiment is noted as KHP-2.

For the field tests on the efficacy of KHP solutions application to the grape plants as disclosed herein, the inventors set up a check group, noted as CK, that is only given water and other normal nutrients; and two control groups, noted as KHP-1 and KHP2, that were additionally applied with the two embodiments of KHP solutions as taught herein.

Three (3) species of grapes were selected for the field tests: Kyoho, Chunfeng and Black Queen, where the first two species are for direct consumption and the Black Queen species being for wine making. Their color changing (ripening) rates and content of anthocyanin are measured and reflected in FIGS. C1 through C6. The figures in C1 and C2 show the result of tests on Kyoho grape; the figures in C3 and C4 show the result of tests on Chunfeng grape; and the figures in C5 and C6 show the test results on Black Queen grape.

FIGS. C1 to C6 show the color changing improvements in accordance with the method of applying KHP solutions disclosed herein.

In FIG. C1A, The KHP-1 and KHP-2 solutions were diluted 500 times before spraying to the leaves' surface (of the Kyoho grape). In weeks 15 to 20, the color changing measurements were taken weekly and noted. As shown, both solution-applied groups promote earlier color changing than the check group, especially from week 15 to week 18, allowing shortened period of time for harvesting.

In FIG. C1B, the color changing rate is specifically measure at 16^{th} week. As shown both solution-applied groups have substantially higher better coloring and thus commanding better price premium.

In FIG. C2A, the Kyoho grape's skins were collected weekly from week 15 to week 22 and measurements of the anthocyanin were taken. As shown, both solution-applied groups produced higher content of anthocyanin.

In FIG. C2B, the anthocyanin content is specifically measured at 17^{th} week. The bar charts clearly show that the check group has much lower amount of anthocyanin than the two KHP solution groups.

In FIG. C3A, The KHP-1 and KHP-2 solutions were diluted 500 times before spraying to the leaves' surface (of the Chunfeng grape). In weeks 13-18, the color changing measurements were taken weekly and noted. As shown, both solution-applied groups promote earlier color changing than the check group, especially from week 14 to week 16, allowing shortened period of time for harvesting.

In FIG. C3B, the anthocyanin content is specifically measured at 15^{th} week. The bar charts clearly show that the check group has much lower amount of anthocyanin than the two KHP solution groups.

In FIG. C4A, the Chunfeng grape's skins were collected weekly from week 14 to week 18 and measurements of the anthocyanin were taken. As shown, both solution-applied groups produced higher content of anthocyanin.

In FIG. C4B, the anthocyanin content from the skins of Chunfeng grape is specifically measured at 17^{th} week. The bar charts clearly show that the check group has much lower amount of anthocyanin than the two KHP solution groups.

In FIG. C5A, The KHP-1 and KHP-2 solutions were diluted 500 times before spraying to the leaves' surface (of the Black Queen grape). In weeks 16-20, the color changing measurements were taken weekly and noted. As shown, both solution-applied groups promote earlier color changing than the check group, especially from week 16 to week 19, allowing shortened period of time for harvesting.

In FIG. C5B, the color changing rate is specifically measured at 18^{th} week. The bar charts clearly show that the two KHP groups have better color changing rate than the check group.

In FIG. C6A, the Black Queen grape's skins were collected weekly from week 15 to week 22 and measurements of the anthocyanin were taken. As shown, both solution-applied groups produced higher content of anthocyanin.

In FIG. C6B, the anthocyanin content is specifically measured at 17^{th} week. The bar charts clearly show that the check group has much lower amount of anthocyanin than the two KHP solution groups.

FIGS. F1 to F5 show the flavor, and crispness, improvements in accordance with the method of applying KHP solutions disclosed herein.

Around harvest time, the Kyoho grape's skins were taken and measured to get the readings of phenolic content. FIG. F1A shows that both KHP solution groups show substantially higher phenolic content than the check (CK) group.

Around harvest time, the Kyoho grapes juiced were taken and measured to get the readings of phenolic content. FIG. F1B shows that both KHP solution groups show substantially higher phenolic content than the check (CK) group.

Around harvest time, the Kyoho grape's skins were taken and measured to get the readings of crispness and elasticity. FIG. F2A shows bar chats on crispness and FIG. F2B shows the bar charts on elasticity. There is no discernible difference between the KHP solution groups and the check (CK) group.

Around harvest time, the Chunfeng grape's skins were taken and measured to get the readings of phenolic content. FIG. F3A shows that both KHP solution groups show substantially higher phenolic content than the check (CK) group.

Around harvest time, the Chunfeng grapes juices were taken and measured to get the readings of phenolic content. FIG. F3B shows that both KHP solution groups show substantially higher phenolic content than the check (CK) group.

Around harvest time, the Chenfeng grape's skins were taken and measured to get the readings of crispness and elasticity. FIG. F4A shows bar chats on crispness and FIG. F4B shows the bar charts on elasticity. The KHP solution groups' crispness seems to be better while the elasticity reading did not fare better.

Around harvest time, the Black Queen grape's skins were taken and measured to get the readings of phenolic content. FIG. F5A shows that both KHP solution groups show substantially higher phenolic content than the check (CK) group.

Around harvest time, the Black Queen grapes juices were taken and measured to get the readings of phenolic content. FIG. F5B shows that both KHP solution groups show substantially higher phenolic content than the check (CK) group.

FIGS. S1 to S3 show the sweetness improvement, including the Brix/Acidity ratio, in accordance with the method of applying KHP solutions disclosed herein. The tests were done on the grape juices by a Sweetness analyzer (PAL-BX/ACID2, Atago) to obtain the measured numbers.

Around harvest time, the Kyoho grapes juices were taken to get the readings of the Brix number. FIG. S1A shows the Brix numbers of the three groups. FIG. S1B show the acidity numbers. FIG. S1C shows the Brix/Acidity ratio.

Around harvest time, the Chenfeng grapes juices were taken to get the readings of the Brix number. FIG. S2A shows the Brix numbers of the three groups. FIG. S2B show the acidity numbers. FIG. S2C shows the Brix/Acidity ratio.

Around harvest time, the Black Queen grapes juices were taken to get the readings of the Brix number. FIG. S3A shows the Brix numbers of the three groups. FIG. S3B show the acidity numbers. FIG. S3C shows the Brix/Acidity ratio.

As can be shown by the Figures in S1 through S3, the sweetness reading and the sweetness to acidity (sourness) all are improved greatly in the KHP solution groups, as compared to the check group.

Overall, the research and experiment show that the method of making the KHP solutions and the application substantially increased the color changing rate, the flavor and the sweetness of grape fruits, in both direct consumption and in the wine-making species.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. The method of using a keratin hydrolysis peptide (KHP) solution to improve grape plant's ripening, flavor and sweetness, comprising the steps of:
a. mixing 40 kg of water and 50 kg of feathers together, wherein the feathers are of 50% water content;
b. stirring the feathers and water mixture in a sealed container;
c. hydrolyzing the mixture in the container with a temperature and pressure setting of 185° C. and 12 kg/cm² for a duration of 80 minutes;
d. using a mass spectrometer to confirm the combination of peptides in the solution to contain at least 253 peptides as listed in the specification whereby their molecular masses are between 500 and 4,000 Dalton, and consisting of between 5 and 30 amino acids;
e. the solution is further filtered and concentrated to the range of 300,000 450,000 ppm concentration;
f. Applying water to dilute the solution to grape leaves' surface during a grape plant's fruit expansion stage.

2. The method of claim 1 wherein the solution is applied to grape leaves' surface during the grape plant's fruit thinning stage.

3. The method of claim 1 wherein the amount of water used to dilute the solution has a volume ratio of 125-1,000 (water):1 (solution).

4. The method of claim 1 wherein the amount of water used to dilute the solution has a volume ratio of 300-500 (water):1 (solution).

5. The method of claim 2 wherein the amount of water used to dilute the solution has a volume ratio of 125-1,000 (water):1 (solution).

6. The method of claim 2 wherein the amount of water used to dilute the solution has a volume ratio of 300-500 (water):1 (solution).

7. The method of using a keratin hydrolysis peptide (KHP) solution to improve grape plant's ripening, flavor and sweetness, comprising the steps of:
a. putting 70 kg of feathers in a sealed container, wherein the feathers are of 46% water content;
b. hydrolyzing the feathers in the container with a temperature and pressure setting of 180° C. and 13 kg/cm² for a duration of 40 minutes;
c. using a mass spectrometer to confirm the combination of peptides in the solution to contain at least 253 peptides as listed in the specification whereby their molecular masses are between 500 and 4,000 Dalton, and consisting of between 5 and 30 amino acids;
d. the solution is further filtered and concentrated to the range of 300,000-450,000 ppm concentration;
e. Applying water to dilute the solution to grape leaves' surface during a grape plant's fruit expansion stage.

8. The method of claim 7 wherein the solution is applied to grape leaves' surface during a grape plant's fruit thinning stage.

9. The method of claim 7 wherein the amount of water used to dilute the solution has a volume ratio of 125-1,000 (water):1 (solution).

10. The method of claim 7 wherein the amount of water used to dilute the solution has a volume ratio of 300-500 (water):1 (solution).

11. The method of claim 8 wherein the amount of water used to dilute the solution has a volume ratio of 125-1,000 (water):1 (solution).

12. The method of claim 8 wherein the amount of water used to dilute the solution has a volume ratio of 300-500 (water):1 (solution).
